(19) United States
(12) Patent Application Publication
(10) Pub. No.: US 2010/0203097 A1
(43) Pub. Date: Aug. 12, 2010

(54) Foundation Compositions Comprising Water Repelling Silicone Elastomer Powders

(76) Inventor: Kojo Tanaka, Ashiya (JP)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/701,125

(22) Filed: Feb. 5, 2010

Related U.S. Application Data
(60) Provisional application No. 61/150,438, filed on Feb. 6, 2009.

(51) Int. Cl. A61K 8/02; A61K 8/84; A61Q 1/00

(52) U.S. Cl. 424/401; 424/63

(57) ABSTRACT

Cosmetic foundation compositions comprising: (a) from about 0.1% to about 85% of a water repelling silicone elastomer powder comprising 100 weight parts of a spherical silicone elastomer particle and 0.5-25 weight parts of polyorganosilsequioxane for coating the spherical silicone elastomer particle; wherein the water repelling silicone elastomer powder does not disperse in, but floats in water; has an average particle size of at least 1 μm and has a softness of from about 10 to about 80 measured by Durometer A Hardness; and (b) a suitable carrier, the suitable carrier comprising from about 0.1% to about 99.8% by weight of the composition of a powder component.

### CROSS REFERENCE TO RELATED APPLICATIONS

This application claims the benefit of U.S. Provisional Application No. 61/150,438 filed on Feb. 6, 2009.

### FIELD OF THE INVENTION

The present invention relates to foundation compositions comprising water repelling silicone elastomer powders.

### BACKGROUND

A foundation composition can be applied to the face and other parts of the body to even skin tone and texture and to hide pores, imperfections, fine lines and the like. A foundation composition is also applied to moisturize the skin, to balance the oil level of the skin, and to provide protection against the adverse effects of sunlight, wind, and other environmental factors.

Foundation compositions are generally available in the form of liquid or cream suspensions, emulsions, gels, pressed powders, loose powders or anhydrous oil and wax compositions. Emulsion-type foundations are suitable in that they provide moisturizing effects by the water and water-soluble skin treatment agents incorporated. On the other hand, a larger amount and variation of powders and pigments can be formulated into pressed powders and loose powders.

Recently, the demanding consumers seek, as functions of a foundation, good feel upon application as well as the ideal look having both good coverage and natural look on the skin. Spherical and translucent powders such as silicone elastomers can improve the natural appearance by light diffusion effect due to its shape and translucency, and also provide good smooth feel. While these materials are highly useful in foundation compositions, they are not easily formulated at a high level, as powder components and pigments which provide other benefits such as wear would be compromised. There is also a need for silicone elastomers which can be formulated over a wide range of foundation compositions, from liquids to loose powders.

Based on the foregoing, there is a need for a foundation composition which provides improved wear benefits, while maintaining natural appearance and good smooth feel.

None of the existing art provides all of the advantages and benefits of the present invention.

### SUMMARY

The present invention is directed to a cosmetic foundation composition comprising:
(a) from about 0.1% to about 85% of a water repelling silicone elastomer powder comprising 100 weight parts of a spherical silicone elastomer particle and 0.5-25 weight parts of polyorganosilsequioxane for coating the spherical silicone elastomer particle; wherein the water repelling silicone elastomer powder does not disperse in, but floats in water; has an average particle size of at least 1 μm and has a softness of from about 10 to about 80 measured by Durometer A Hardness; and
(b) a suitable carrier, the suitable carrier comprising from about 0.1% to about 99.8% by weight of the composition of a powder component.

These and other features, aspects, and advantages of the present invention will become evident to those skilled in the art from a reading of the present disclosure with the appended claims

### BRIEF DESCRIPTION OF THE DRAWINGS

While the specification concludes with claims particularly pointing out and distinctly claiming the invention, it is believed that the present invention will be better understood from the following description of preferred, nonlimiting embodiments and representations taken in conjunction with the accompanying drawings in which:

FIG. 1 is a microscopic photograph of a preferred embodiment of a collapsible water-containing capsule product form foundation of the present invention, along with a scale showing the length of 100 μm.

### DETAILED DESCRIPTION

While the specification concludes with claims particularly pointing out and distinctly claiming the invention, it is believed that the present invention will be better understood from the following description.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

All ingredients such as actives and other ingredients useful herein may be categorized or described by their cosmetic and/or therapeutic benefit or their postulated mode of action. However, it is to be understood that the active and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

### Water Repelling Silicone Elastomer Powder

The present composition comprises, by weight of the composition, from about 0.1% to about 85% of a water repelling silicone elastomer powder. The water repelling silicone elastomer powder herein has a particle size of at least 1 μm, preferably from about 1 μm to about 25 μm, more preferably from about 4 μm to about 15 μm, and is spherical in shape. Without being bound by theory, it is believed that, by the highly hydrophobic surface, relatively large size and spherical shape of the water repelling silicone elastomer powder, improved wear benefits, while maintaining natural appearance and good smooth feel are provided to the composition. Further, for product forms that are water-containing capsules, it is believed that the smaller size powder component surround the water phase to make a first layer, and the larger size water repelling silicone elastomer powder aligns at the phase boundary of the smaller size color powder, and provides good smooth feel and improved stability to the overall capsule. The water repelling silicone elastomer powder can improve the natural appearance by light diffusion effect due to its shape and translucency, and may also alleviate negative skin feel that some other smaller size color powders may cause.

The water repelling silicone elastomer powder herein comprises 100 weight parts of a spherical silicone elastomer particle and 0.5-25 weight parts of polyorganosilsequioxane for coating the spherical silicone elastomer particle; wherein the water repelling silicone elastomer powder does not disperse in, but floats in water; has an average particle size of at least 1 μm and has a softness of from about 10 to about 80 measured by Durometer A Hardness, preferably the surface of the coated polyorganosilsequioxane is further bonded with a trimethylsilyl group, and preferably surface of the coated polyorganosilsequioxane is further condensated by hydrolyzing with tetraalkoxysilane and at least one silylation agent selected from the group consisting of trimethylalkoxysilane, trimethylsilanol, and hexamethyldisilazine. Such water repelling silicone elastomer powder is particularly advantageous for providing stability to the capsule. Such water repelling silicone elastomer powder is exemplified as Reference Examples 1 and 2 below.

### Powder Component

The composition of the present composition comprises, by weight of the composition, from about 0.1% to about 99.8% of a powder component. The amount of powder component included in each product form is described in the sections below. The powder component herein can be of any size that is used for cosmetic foundations, however, when used for water-containing capsules, the powder components must have a particle size of from about 4 nm to less than 1 μm, preferably from about 5 nm to about 500 nm, and is surface coated with a hydrophobic coating material. The species and levels of the powders herein provide, for example, shade, coverage, UV protection benefit, good wear performance, and stability in the composition. Depending on the needs of the product, colorless powders may be selected for providing a colorless foundation, and/or a make up base composition.

Powder components useful for the powder component herein are clay mineral powders such as talc, mica, sericite, silica, magnesium silicate, synthetic fluorphlogopite, calcium silicate, aluminum silicate, bentonite and montmorillonite; pearl powders such as alumina, barium sulfate, calcium secondary phosphate, calcium carbonate, titanium dioxide, finely divided titanium dioxide, zirconium oxide, zinc oxide, hydroxy apatite, iron oxide, iron titanate, ultramarine blue, Prussian blue, chromium oxide, chromium hydroxide, cobalt oxide, cobalt titanate, titanium dioxide coated mica; organic powders such as polyester, polyethylene, polystyrene, methyl methacrylate resin, cellulose, 12-nylon, 6-nylon, styrene-acrylic acid copolymers, polypropylene, vinyl chloride polymer, tetrafluoroethylene polymer, boron nitride, fish scale guanine, laked tar color dyes, and laked natural color dyes. Such powders may be treated with a hydrophobical treatment agent, including: silicone such as Methicone, Dimethicone and perfluoroalkylsilane; fatty material such as stearic acid; metal soap such as aluminum dimyristate; aluminum hydrogenated tallow glutamate, hydrogenated lecithin, lauroyl lysine, aluminum salt of perfluoroalkyl phosphate, and mixtures thereof.

The powder components useful herein include those that provide color or change tone, and also those that provide a certain skin feel. Useful pigments herein include clay mineral powders such as silica, talc, magnesium silicate, synthetic fluorphlogopite, calcium silicate, boron nitride, aluminum silicate, bentonite and montomorilonite. The coloring powders useful herein include pearl pigments such as alumina, barium sulfate, calcium secondary phosphate, zirconium oxide, zinc oxide, hydroxy apatite, iron oxide, iron titate, ultramarine blue, Prussian blue, chromium oxide, chromium hydroxide, cobalt oxide, cobalt titanate, titanium dioxide coated mica; organic powders such as polyester, polyethylene, polystyrene, methyl metharylate resin, 12-nylon, 6-nylon, styrene-acrylic acid copolymers, poly propylene, vinyl chloride polymer, tetrafluoroethylene polymer, fish scale guanine, laked tar color dyes, and laked natural color dyes. Particularly useful herein as the powder component are titanium dioxide, zinc oxide, iron oxide, barium sulfate, silica, and mixtures thereof.

The powder components are preferably coated with a coating material having hydrophobic characteristics. Useful hydrophobic coating materials herein include methyl polysiloxane, methyl hydrogen polysiloxane, methyl phenyl polysilxoane, n-octyl triethoxy silane, methyl-alpha-styrene polysiloxane, acryl silicone copolymer, and mixtures thereof.

One highly preferred powder component herein for collapsible water-containing capsule compositions is a spindle-shaped metal oxide powder which is hydrophobically surface-treated and has an average long axis particle size of from about 25 nm to about 150 nm, preferably from about 30 nm to about 100 nm, an average short axis particle size of from about 4 nm to about 50 nm, preferably from about 5 nm to about 20 nm, and an aspect ratio of greater than about 3, preferably greater than about 4. The metal oxide is preferably selected from titanium oxide, zinc oxide and iron oxide, more preferably titanium dioxide. The coating materials useful for hydrophobic surface-treating of the spindle-shaped metal oxide powder include dimethyl polysiloxane, methyl hydrogen polysiloxane, methyl phenyl polysiloxane, n-octyl triethoxy silane, methyl-alpha-styrene polysiloxane, acryl silicone copolymer, and mixtures thereof. Without being bound by theory, it is believed that, by the surface tension of the hydrophobic surface of the spindle-shaped metal oxide powder, the spindle-shaped metal oxide powders align at the phase boundary of the water phase binding with each other via van-der-Waals binding, while the high aspect ratio shape provides a fractal structure surrounding and repelling the water phase. It is further believed that the overall structure due to the hydrophobic surface, combined with the relatively small particle size of the spindle-shaped metal oxide powder, contributes to the suitable shear stress tolerance of the composition of the present composition.

Commercially available filler powders herein include Titanium Dioxide coated with triethoxycaprylylsilane having a long axis particle size of about 60 nm and a short axis particle size of about 10 nm (aspect ratio about 6) with tradename OTS-11 TTO-V-3 available from Daito Kasei, silica dimethyl silylate having a particle size of 15 nm with tradename Aerosil R972 available from Nihon Aerosil, Titanium Dioxide coated with triethoxycaprylsilane having a particle size of about 250 nm with tradename OTS-2 TIO2 CR-50 available from Daito Kasei,

Zinc Oxide coated with Triethoxycaprylylsilane having a particle size of about 20 nm with tradename OTS-7 FZO-50 available from Daito Kasei, Mica, Titanium Dioxide coated with Dimethicone having a particle size of about 20 μm with tradename SA FLAMENCO RED available from Miyoshi Kasei, yellow, black and red iron oxide coated with Triethoxycaprylylsilane having an particle size of about 400 nm with tradenames OTS-2 YELLOW LL-100P, OTS-2 BLACK BL-100P, and OTS-2 RED R-516P available from Daito Kasei.

### Suitable Carrier and Product Forms

The combination of water repelling silicone elastomer powder and powder component of the present invention can be incorporated in foundations of various product forms, while minimizing the affect to other benefits of the composition. Suitable product forms include collapsible water-containing capsule, loose powder, pressed powder, water-in-oil emulsion, oil-in-water emulsion, and solid forms of such emulsions. Respective product forms and their respective suitable carriers are listed hereinbelow.

For providing collapsible water-containing capsule forms, the carrier comprises, by weight of the composition:
(a) from about 0.1 to about 60% of a powder component wherein the total of the water repelling silicone elastomer powder and the powder component is at least 5%; and
(b) from about 40% to about 95% of a water phase.

For providing loose powder forms, the carrier comprises, by weight of the composition, from about 15% to about 99.8% of a powder component.

For providing pressed powder forms, the carrier comprises, by weight of the composition:
(a) from about 55% to about 98.9% of a powder component; and
(b) from about 1% to about 25% of a liquid binder selected from a water phase, a liquid oil, a water-in-oil emulsifier, and mixtures thereof.

For providing water-in-oil emulsion forms that are liquid or paste, the carrier comprises, by weight of the composition:
(a) from about 5% to about 60% of an oil component;
(b) from about 0.1% to about 25% of a water-in-oil emulsifier; and
(c) from about 5% to about 70% of a water phase.

For providing oil-in-water emulsion forms that are liquid or paste, the carrier comprises, by weight of the composition:
(a) from about 5% to about 60% of an oil component;
(b) from about 0.1% to about 25% of a oil-in-water emulsifier; and
(c) from about 10% to about 85% of a water phase.

For providing water-in-oil or oil-in-water emulsions that are in solid form, the carrier further comprises, by weight of the composition, from about 0.1% to about 10% of a solid wax.

In one highly preferred embodiment, the composition is a collapsible water-containing capsule which comprises, by weight of the capsule, from about 40% to about 95% of a water phase. To hold such abundant amount of water in the structure, the capsule of the present invention comprises the water repelling silicone elastomer powder and the powder component. By providing at least 5% of the total of water repelling silicone elastomer powder and the powder component, the capsule is provided with stability under normal storage conditions as well as normal mixing processes, however, collapses upon application. Without being bound by theory, it is believed that the smaller size submicron powder components surround the water phase to make a first layer, the larger size water repelling silicone elastomer powder provides a second layer on top of the submicron powder components, while the water repelling silicone elastomer powder also acts as a spacer for maintaining balanced adhesion with each other, and thereby provide the stability and integrity of the capsule. It is believed that the dual covered structure provided by the submicron powder component and water repelling silicone elastomer powders provide improved shear stress tolerance of the collapsible water-containing capsule of the present composition.

### Water Phase

Depending on the product form, the composition of the present invention may comprise a water phase, the water phase comprising water, optional water-soluble solvent, and optional gelling agent, detailed hereafter. The water phase may be made only by water. Deionized water is preferably used. Water from natural sources including mineral cations can also be used, depending on the desired characteristic of the product. In one preferred embodiment, water may be sourced from fermented biological cultures or its filtrates. A highly preferred commercial source of this kind is Galactomyces ferment filtrate by the tradename SK-II Pitera available from Kashiwayama.

The pH of the water phase is selected in view of the desired characteristic of the product, and particularly, when skin benefit agents are included, the activity and stability of the skin benefit agents. In one preferred embodiment the pH is adjusted to from about 4 to about 8. Buffers and other pH adjusting agents can be included to achieve the desirable pH.

### Water-Soluble Solvent

The water phase of the composition of the present invention may further comprise a water-soluble solvent selected from lower alkyl alcohols and water-soluble humectants. The water-soluble solvents are selected according to the desired skin feel to be delivered, and/or for delivering certain skin benefit agents.

Lower alkyl alcohols useful herein are monohydric alcohols having 1 to 6 carbons, more preferably ethanol and isopropanol.

Water soluble humectants useful herein include polyhydric alcohols such as butylene glycol(1,3 butanediol), pentylene glycol(1,2-pentanediol), glycerin, sorbitol, propylene glycol, hexylene glycol, ethoxylated glucose, 1,2-hexane diol, hexanetriol, dipropylene glycol, erythritol, trehalose, diglycerin, xylitol, maltitol, maltose, glucose, fructose; and other water-soluble compounds such as urea, sodium chondroitin sulfate, sodium hyaluronate, sodium adenosin phosphate, sodium lactate, pyrrolidone carbonate, cyclodextrin, and mixtures thereof. Also useful herein include water soluble alkoxylated nonionic polymers such as polyethylene glycols and polypropylene glycols having a molecular weight of up to about 1000 such as those with CTFA names PEG-200, PEG-400, PEG-600, PEG-1000, and mixtures thereof.

In one preferred embodiment, the present composition comprises from about 1% to about 30% of a water-soluble humectant. In one highly preferred embodiment wherein the composition is used as a foundation, the composition comprises from about 3% to about 30% of a water-soluble humectant.

Commercially available humectants herein include: butylene glycol with tradename 1,3-Butylene glycol available from Celanese, pentylene glycol with tradename HYDROLITE-5 available from Dragoco, glycerin with tradenames STAR and SUPEROL available from The Procter & Gamble Company, CRODEROL GA7000 available from Croda Universal Ltd., PRECERIN series available from Unichema, and a same tradename as the chemical name available from NOF; propylene glycol with tradename LEXOL PG-865/855 available from Inolex, 1,2-PROPYLENE GLYCOL USP available from BASF; sorbitol with tradenames LIPONIC series available from Lipo, SORBO, ALEX, A-625, and A-641 available from ICI, and UNISWEET 70, UNISWEET CONC available from UPI; dipropylene glycol with the same tradename available from BASF; diglycerin with tradename DIGLYCEROL available from Solvay GmbH; xylitol with the same tradename available from Kyowa and Eizai; maltitol with tradename MALBIT available from Hayashibara, sodium chondroitin sulfate with the same tradename available from Freeman and Bioiberica, and with tradename ATOMERGIC SODIUM CHONDROITIN SULFATE available from Atomergic Chemetals; sodium hyaluronate available from Chisso Corp, the same with tradenames ACTIMOIST available from Active Organics, AVIAN SODIUM HYALURONATE series available from Intergen, HYALURONIC ACID Na available from Ichimaru Pharcos; sodium adenosin phophate with the same tradename available from Asahikasei, Kyowa, and Daiichi Seiyaku; sodium lactate with the same tradename available from Merck, Wako, and Showa Kako, cyclodextrin with tradenames CAVITRON available from American Maize, RHODOCAP series available from Rhone-Poulenc, and DEXPEARL available from Tomen; and polyethylene glycols with the tradename CARBOWAX series available from Union Carbide.

### Gelling Agents

The water phase of the composition of the present composition may further comprise, by weight of the composition, from about 0.1% to about 20%, preferably from about 0.1% to about 5%, of a gelling agent that provides the water phase a viscosity of from about 10 mPas to about 1,000,000 mPas, preferably from about 10 mPas to about 100,000 mPas.

The polymers useful as the gelling agent herein are water soluble or water miscible polymers. The term “water soluble or water miscible” with regard to the gelling agents herein, relate to compounds that are dissolved to make a transparent solution when dissolved in ample amount of water with or without the aid of elevated temperature and/or mixing.

Useful herein are starch derivative polymers such as carboxymethyl starch, and methylhydroxypropyl starch. Commercially available compounds that are highly useful herein include sodium carboxymethyl starch with tradename COVAGEL available from LCW.

Useful herein are cellulose derivative polymers. Cellulose derivative polymers useful herein include methylcellulose, ethylcellulose, hydroxyethylcellulose, hydroxyethyl ethylcellulose, hydroxypropyl methyl cellulose, nitrocellulose, sodium cellulose sulfate, sodium carboxymethylcellulose, crystalline cellulose, cellulose powder, and mixtures thereof. Also useful are starch derivative polymers such as carboxymethyl starch, and methylhydroxypropyl starch. Commercially available compounds that are highly useful herein include hydroxyethylcellulose with tradename Natrosol Hydroxyethylcellulose, and carboxymethylcellulose with tradename Aqualon Cellulose Gum, both available from Aqualon.

Useful herein are carboxylic acid/carboxylate copolymers. Commercially available carboxylic acid/carboxylate copolymers useful herein include: CTFA name Acrylates/C10-30 Alkyl Acrylate Crosspolymer having tradenames Pemulen TR-1, Pemulen TR-2, Carbopol 1342, Carbopol 1382, and Carbopol ETD 2020, all available from B. F. Goodrich Company.

Neutralizing agents may be included to neutralize the carboxylic acid/carboxylate copolymers herein. Nonlimiting examples of such neutralizing agents include sodium hydroxide, potassium hydroxide, ammonium hydroxide, monoethanolamine, diethanolamine, triethanolamine, diisopropanolamine, aminomethylpropanol, tromethamine, tetrahydroxypropyl ethylenediamine, and mixtures thereof.

Polyalkylene glycols having a molecular weight of more than about 1000 are useful herein. Useful are those having the following general formula:

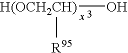

wherein R⁹⁵ is selected from the group consisting of H, methyl, and mixtures thereof. When R⁹⁵ is H, these materials are polymers of ethylene oxide, which are also known as polyethylene oxides, polyoxyethylenes, and polyethylene glycols. When R⁹⁵ is methyl, these materials are polymers of propylene oxide, which are also known as polypropylene oxides, polyoxypropylenes, and polypropylene glycols. When R⁹⁵ is methyl, it is also understood that various positional isomers of the resulting polymers can exist. In the above structure, x3 has an average value of from about 1500 to about 25,000, preferably from about 2500 to about 20,000, and more preferably from about 3500 to about 15,000. Other useful polymers include the polypropylene glycols and mixed polyethylene-polypropylene glycols, or polyoxyethylene-polyoxypropylene copolymer polymers. Polyethylene glycol polymers useful herein are PEG-2M wherein R⁹⁵ equals H and x3 has an average value of about 2,000 (PEG-2M is also known as Polyox WSR® N-10, which is available from Union Carbide and as PEG-2,000); PEG-5M wherein R⁹⁵ equals H and x3 has an average value of about 5,000 (PEG-5M is also known as Polyox WSR® N-35 and Polyox WSR® N-80, both available from Union Carbide and as PEG-5,000 and Polyethylene Glycol 300,000); PEG-7M wherein R⁹⁵ equals H and x3 has an average value of about 7,000 (PEG-7M is also known as Polyox WSR® N-750 available from Union Carbide); PEG-9M wherein R⁹⁵ equals H and x3 has an average value of about 9,000 (PEG 9-M is also known as Polyox WSR® N-3333 available from Union Carbide); and PEG-14 M wherein R⁹⁵ equals H and x3 has an average value of about 14,000 (PEG-14M is also known as POLYOX WSR® N-3000 available from Union Carbide).

Useful herein are vinyl polymers such as cross linked acrylic acid polymers with the CTFA name Carbomer, pullulan, mannan, scleroglucans, polyvinylpyrrolidone, polyvinyl alcohol, guar gum, hydroxypropyl guar gum, xanthan gum, acacia gum, arabia gum, tragacanth, galactan, carob gum, karaya gum, locust bean gum, carrageenin, pectin, amylopectin, agar, quince seed (Cydonia oblonga Mill), starch (rice, corn, potato, wheat), algae colloids (algae extract), microbiological polymers such as dextran, succinoglucan, starch-based polymers such as carboxymethyl starch, methylhydroxypropyl starch, alginic acid-based polymers such as sodium alginate, alginic acid propylene glycol esters, acrylate polymers such as sodium polyacrylate, polyacrylamide, polyethyleneimine, and inorganic water soluble material such as bentonite, aluminum magnesium silicate, laponite, hectonite, and anhydrous silicic acid.

Commercially available gelling agents useful herein include xanthan gum with tradename KELTROL series available from Kelco, Carbomers with tradenames CARBOPOL 934, CARBOPOL 940, CARBOPOL 950, CARBOPOL 980, and CARBOPOL 981, all available from B. F. Goodrich Company, acrylates/steareth-20 methacrylate copolymer with tradename ACRYSOL 22 available from Rohm and Hass, polyacrylamide with tradename SEPIGEL 305 available from Seppic, sodium polyacrylate with tradename COVACRYL MV60 available from LCW, glyceryl polymethacrylate with tradename LUBRAGEL NP, and a mixture of glyceryl polymethacrylate, propylene glycol and PVM/MA copolymer with tradename LUBRAGEL OIL available from ISP, scleroglucan with tradename Clearogel SC11 available from Michel Mercier Products Inc. (NJ, USA), ethylene oxide and/or propylene oxide based polymers with tradenames CARBOWAX PEGs, POLYOX WASRs, and UCON FLUIDS, all supplied by Amerchol.

Useful herein are amphoteric polymers such as Polyquaternium 22 with tradenames MERQUAT 280, MERQUAT 295, Polyquaternium 39 with tradenames MERQUAT PLUS 3330, MERQUAT PLUS 3331, and Polyquaternium 47 with tradenames MERQUAT 2001, MERQUAT 2001N, all available from Calgon Corporation. Other useful amphoteric polymers include octylacrylamine/acrylates/butylaminoethyl methacrylate copolymers with the tradenames AMPHOMER, AMPHOMER SH701, AMPHOMER 28-4910, AMPHOMER LV71, and AMPHOMER LV47 supplied by National Starch & Chemical.

### Oil Components

Depending on the product form, the present composition may comprise, as a suitable carrier, an oil component selected from volatile silicone oil, non-volatile oil, thickeners, and mixtures thereof.

Useful for the present invention is a volatile silicone oil. When incorporated in water-in-oil emulsions, preferably, the amount of the volatile silicone oil is controlled so that the composition comprises from about 20% to about 50% of the volatile silicone oil, and the total of the volatile silicone oil and water is more than about 50% of the entire composition. Without being bound by theory, the species and levels of the volatile silicone oil herein is believed to provide improved refreshing and light feeling to the skin, without necessarily leaving a dried feeling to the skin. Volatile silicone oils can also be used as a binder for powder forms of the present composition.

The volatile silicone oil useful herein are selected from those having a boiling point of from about 60 to about 260° C., preferably those having from 2 to 7 silicon atoms.

The volatile silicone oils useful herein include polyalkyl or polyaryl siloxanes with the following structure (I):

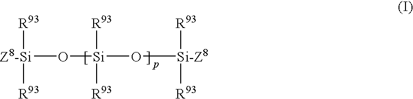

wherein R⁹³ is independently alkyl or aryl, and p is an integer from about 0 to about 5. Z⁸ represents groups which block the ends of the silicone chains. Preferably, R⁹³ groups include methyl, ethyl, propyl, phenyl, methylphenyl and phenylmethyl, Z⁸ groups include hydroxy, methyl, methoxy, ethoxy, propoxy, and aryloxy. More preferably, R⁹³ groups and Z⁸ groups are methyl groups. The preferred volatile silicone compounds are hexamethyldisiloxane, octamethyltrisiloxane, decamethyltetrasiloxane, hexadecamethylheptasiloxane. Commercially available volatile silicone compounds useful herein include octamethyltrisiloxane with tradename SH200C-1cs, decamethyltetrasiloxane with tradename SH200C-1.5cs, hexadecamethylheptasiloxane with tradename SH200C-2cs, all available from Dow Corning.

The volatile silicone oils useful herein also include a cyclic silicone compound having the formula:

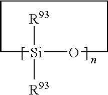

wherein R⁹³ is independently alkyl or aryl, and n is an integer of from 3 to 7.

Preferably, R⁹³ groups include methyl, ethyl, propyl, phenyl, methylphenyl and phenylmethyl. More preferably, R⁹³ groups are methyl groups. The preferred volatile silicone compounds are octamethylcyclotetrasiloxane, decamethylcyclopentasiloxane, tetradecamethylcyclohexasiloxane. Commercially available volatile silicone compounds useful herein include octamethylcyclotetrasiloxane with tradename SH244, decamethylcyclopentasiloxane with tradename DC245 and SH245, and dodeamethylcyclohexasiloxane with tradename DC246; all available from Dow Corning.

Useful for the composition of the present invention comprises a non-volatile oil. When incorporated in water-in-oil emulsions, preferably, the amount is from about 0.5% to about 20%. When the emulsion is made into solid form, preferably, the amount is from about 0.5% to about 10%. Without being bound by theory, the species and levels of the non-volatile oil herein is believed to provide improved smoothness to the skin, and also alleviate dry feeling of the skin. Non-volatile oils can also be used as a binder for powder forms of the present composition.

Non-volatile oils useful herein are, for example, tridecyl isononanoate, isostearyl isostearate, isocetyl isosteatrate, isopropyl isostearate, isodecyl isonoanoate, cetyl octanoate, isononyl isononanoate, diisopropyl myristate, isocetyl myristate, isotridecyl myristate, isopropyl myristate, isostearyl palmitate, isocetyl palmitate, isodecyl palmitate, isopropyl palmitate, octyl palmitate, caprylic/capric acid triglyceride, glyceryl tri-2-ethylhexanoate, neopentyl glycol di(2-ethyl hexanoate), diisopropyl dimerate, tocopherol, tocopherol acetate, avocado oil, camellia oil, turtle oil, macadamia nut oil, corn oil, mink oil, olive oil, rapeseed oil, eggyolk oil, sesame oil, persic oil, wheat germ oil, pasanqua oil, castor oil, linseed oil, safflower oil, cotton seed oil, perillic oil, soybean oil, peanut oil, tea seed oil, kaya oil, rice bran oil, china paulownia oil, Japanese paulownia oil, jojoba oil, rice germ oil, glycerol trioctanate, glycerol triisopalmiatate, trimethylolpropane triisostearate, isopropyl myristate, glycerol tri-2-ethylhexanoate, pentaerythritol tetra-2-ethylhexanoate, lanolin, liquid lanolin, liquid paraffin, squalane, vaseline, and mixtures thereof. Commercially available oils include, for example, tridecyl isononanoate with tradename Crodamol TN available from Croda, Hexalan available from Nisshin Seiyu, and tocopherol acetates available from Eisai.

Non-volatile oils useful herein also include polyalkyl or polyaryl siloxanes with the following structure (I)

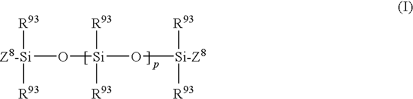

wherein R⁹³ is alkyl or aryl, and p is an integer from about 7 to about 8,000. Z⁸ represents groups which block the ends of the silicone chains. The alkyl or aryl groups substituted on the siloxane chain (R⁹³) or at the ends of the siloxane chains Z⁸ can have any structure as long as the resulting silicone remains fluid at room temperature, is dispersible, is neither irritating, toxic nor otherwise harmful when applied to the skin, is compatible with the other components of the composition, and is chemically stable under normal use and storage conditions. Suitable Z⁸ groups include hydroxy, methyl, methoxy, ethoxy, propoxy, and aryloxy. The two R⁹³ groups on the silicon atom may represent the same group or different groups. Preferably, the two R⁹³ groups represent the same group. Suitable R⁹³ groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company in their Viscasil® and SF 96 series, and from Dow Corning in their Dow Corning 200 series.

Polyalkylaryl siloxane fluids can also be used and include, for example, polymethylphenylsiloxanes. These siloxanes are available, for example, from the General Electric Company as SF 1075 methyl phenyl fluid or from Dow Corning as 556 Cosmetic Grade Fluid.

Non-volatile oils also useful herein are the various grades of mineral oils. Mineral oils are liquid mixtures of hydrocarbons that are obtained from petroleum. Specific examples of suitable hydrocarbons include paraffin oil, mineral oil, dodecane, isododecane, hexadecane, isohexadecane, eicosene, isoeicosene, tridecane, tetradecane, polybutene, polyisobutene, and mixtures thereof.

Useful for the present invention is a thickener. Thickeners can be used for adding viscosity to liquid water-in-oil form compositions, for solidifying solid water-in-oil form compositions, and as a binder for the powder form compositions of the present invention. When used in liquid forms, the thickener is kept to about 5% of the entire composition. The thickeners useful herein are selected from the group consisting of fatty compounds, organic thickeners, inorganic thickeners, and mixtures thereof. The amount and type of thickeners are selected according to the desired viscosity and characteristics of the product.

Fatty compounds useful herein include stearic acid, palmitic acid, stearyl alcohol, cetyl alcohol, behenyl alcohol, stearic acid, palmitic acid, the polyethylene glycol ether of stearyl alcohol or cetyl alcohol having an average of about 1 to about 5 ethylene oxide units, and mixtures thereof. Preferred fatty compounds are selected from stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, and mixtures thereof.

The organic thickeners useful herein include esters and amides of fatty acid gellants, hydroxy acids, hydroxy fatty acids, other amide gellants, and crystalline gellants.

N-acyl amino acid amides useful herein are prepared from glutamic acid, lysine, glutamine, aspartic acid and mixtures thereof. Particularly preferred are n-acyl glutamic acid amides corresponding to the following formula:

R2-NH—CO—(CH2)2-CH—(NH—CO—R1)-CO—NH—R2

wherein R1 is an aliphatic hydrocarbon radical having from about 12 to about 22 carbon atoms, and R2 is an aliphatic hydrocarbon radical having from about 4 to about 12 carbon atoms. Non-limiting examples of these include n-lauroyl-L-glutamic acid dibutyl amide, n-stearoyl-L-glutamic acid diheptyl amide, and mixtures thereof. Most preferred is n-lauroyl-L-glutamic acid dibutyl amide, also referred to as dibutyl lauroyl glutamide. This material is commercially available with tradename Organic thickener GP-1 available from Ajinomoto.

Other organic thickeners suitable for use in the compositions include 12-hydroxystearic acid, esters of 12-hydroxystearic acid, amides of 12-hydroxystearic acid and combinations thereof. These preferred gellants include those which correspond to the following formula:

R1-CO—(CH2)10-CH—(OH)—(CH2)5-CH3

wherein R1 is R2 or NR2R3; and R2 and R3 are hydrogen, or an alkyl, aryl, or arylalkyl radical which is branched linear or cyclic and has from about 1 to about 22 carbon atoms; preferably, from about 1 to about 18 carbon atoms. R2 and R3 may be either the same or different; however, at least one is preferably a hydrogen atom. Preferred among these gellants are those selected from the group consisting of 12-hydroxystearic acid, 12-hydroxystearic acid methyl ester, 12-hydroxystearic acid ethyl ester, 12-hydroxystearic acid stearyl ester, 12-hydroxystearic acid benzyl ester, 12-hydroxystearic acid amide, isopropyl amide of 12-hydroxystearic acid, butyl amide of 12-hydroxystearic acid, benzyl amide of 12-hydroxystearic acid, phenyl amide of 12-hydroxystearic acid, t-butyl amide of 12-hydroxystearic acid, cyclohexyl amide of 12-hydroxystearic acid, 1-adamantyl amide of 12-hydroxystearic acid, 2-adamantyl amide of 12-hydroxystearic acid, diisopropyl amide of 12-hydroxystearic acid, and mixtures thereof; even more preferably, 12-hydroxystearic acid, isopropyl amide of 12-hydroxystearic acid, and combinations thereof. Most preferred is 12-hydroxystearic acid.

Suitable amide gellants include disubstituted or branched monoamide gellants, monosubstituted or branched diamide gellants, triamide gellants, and combinations thereof, excluding the n-acyl amino acid derivatives selected from the group consisting of n-acyl amino acid amides, n-acyl amino acid esters prepared from glutamic acid, lysine, glutamine, apartic acid, and combinations thereof, and which are specifically disclosed in U.S. Pat. No. 5,429,816.

Alkyl amides or di- and tri-basic carboxylic acids or anhydrides suitable for use in the composition include alkyl amides of citric acid, tricarballylic acid, aconitic acid, nitrilotriacetic acid, succinic acid and itaconic acid such as 1,2,3-propane tributylamide, 2-hydroxy-1,2,3-propane tributylamide, 1-propene-1,2,3-triotylamide, N,N′,N″-tri(acetodecylamide)amine, 2-dodecyl-N,N′-dihexylsuccinamide, and 2 dodecyl-N,N′-dibutylsuccinamide. Preferred are alkyl amides of di-carboxylic acids such as di-amides of alkyl succinic acids, alkenyl succinic acids, alkyl succinic anhydrides and alkenyl succinic anhydrides, more preferably 2-dodecyl-N,N′-dibutylsuccinamide.

Inorganic thickeners useful herein include hectorite, bentonite, montmorillonite, and bentone clays which have been modified to be compatible with oil. Preferably, the modification is quaternization with an ammonium compound. Preferable inorganic thickeners include quaternary ammonium modified hectorite. Commercially available oil swelling clay materials include benzyldimethyl stearyl ammonium hectorite with tradename Bentone 38 available from Elementis.

### Water-in-Oil Emulsifier

The water-in-oil emulsion product form composition of the present invention comprises a water-in-oil emulsifier in an amount of preferably from about 0.1% to about 10%. When incorporated in solid water-in-oil emulsion forms, the amount included is preferably from about 1% to about 5%. Without being bound by theory, the species and levels of the water-in-oil emulsifier herein are believed to provide a stable water-in-oil emulsion in view of the other components of the present invention. Water-in-oil emulsifiers can also be used as a binder for powder forms of the present composition. The water-in-oil emulsifier herein has an HLB value of less than about 8.

The HLB value is a theoretical index value which describes the hydrophilicity-hydrophobicity balance of a specific compound. Generally, it is recognized that the HLB index ranges from 0 (very hydrophobic) to 40 (very hydrophilic). The HLB value of the water-in-oil emulsifiers may be found in tables and charts known in the art, or may be calculated with the following general equation: HLB=7+(hydrophobic group values)+(hydrophilic group values). The HLB and methods for calculating the HLB of a compound are explained in detail in Surfactant Science Series, Vol. 1: Nonionic Surfactants”, pp 606-13, M. J. Schick (Marcel Dekker Inc., New York, 1966).

The water-in-oil emulsifier can be an ester-type surfactant. Ester-type surfactants useful herein include: sorbitan monoisostearate, sorbitan diisostearate, sorbitan sesquiisostearate, sorbitan monooleate, sorbitan dioleate, sorbitan sesquioleate, glyceryl monoisostearate, glyceryl diiostearate, glyceryl sesquiisostearate, glyceryl monooleate, glyceryl dioleate, glyceryl sesquioleate, diglyceryl diisostearate, diglyceryl dioleate, diglycerin monoisostearyl ether, diglycerin diisostearyl ether, and mixtures thereof.

Commercially available ester-type surfactants are, for example, sorbitan isostearate having a tradename Crill 6 available from Croda, and sorbitan sesquioleate with tradename Arlacel 83 available from Kao Atras.

The water-in-oil emulsifier can be a silicone-type surfactant. Silicone-type surfactants useful herein are (i), (ii), (iii), and (iv) as shown below, and mixtures thereof.
(i) dimethicone copolyols having the formulation:

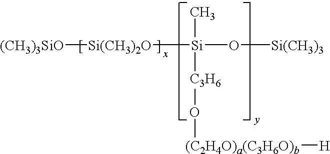

wherein x is an integer from 5 to 100, y is an integer from 1 to 50, a is zero or greater, b is zero or greater, the average sum of a+b being 1-100.
(ii) dimethicone copolyols having the formulation:

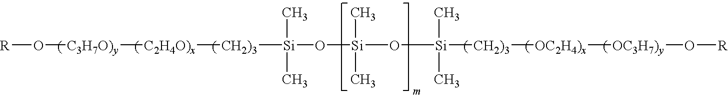

wherein R is selected from the group consisting of hydrogen, methyl, and combinations thereof, m is an integer from 5 to 100, x is independently zero or greater, y is independently zero or greater, the sum of x+y being 1-100.
(iii) branched polyether-polydiorganosiloxane emulsifiers herein having the formulation:

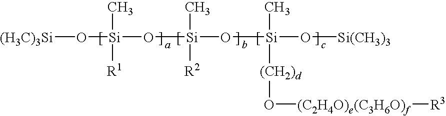

wherein R¹ is an alkyl group having from about 1 to about 20 carbons; R² is

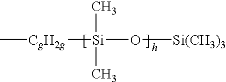

wherein g is from about 1 to about 5, and h is from about 5 to about 20; R³ is H or an alkyl group having from about 1 to about 5 carbons; e is from about 5 to about 20; f is from about 0 to about 10; a is from about 20 to about 100; b is from about 1 to about 15; c is from about 1 to about 15; and d is from about 1 to about 5.
(iv) alkyl dimethicone copolyols which are nonionic polysiloxane copolymer having emulsifying ability, comprising a methylpolysiloxane moiety, an alkyl methylpolysiloxane moiety, and a poly(oxyalkylene)methylpolysiloxane moiety; having an HLB from about 4 to about 6, and a molecular weight of from about 10,000 to about 20,000, wherein the alkyl group is made of from about 10 to about 22 carbons. Suitable alkyl dimethicone copolyols herein are those which have the following formulation:

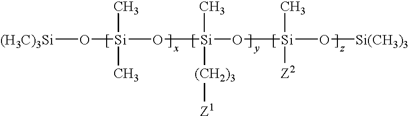

wherein Z¹ is O(C₂H₄O)ₚ(C₃H₆O)_{q}H, p is from 0 to about 50, q is from 0 to about 30, wherein p and q are not 0 at the same time; x is from 1 to about 200, y is from 1 to about 40, and z is from 1 to about 100, and Z² is an alkyl group having from about 10 to about 22 carbons, preferably from about 16 to about 18 carbons. Commercially available silicone-type surfactants are, for example, dimethicone copolyols DC5225C, BY22-012, BY22-008, SH3746M, SH3771M, SH3772M, SH3773M, SH3775M, SH3748, SH3749, and DC5200, all available from Dow Corning, and branched polyether-polydiorganosiloxane emulsifiers such as PEG-9 polydimethylsiloxyethyl Dimethicone, having an HLB of about 4 and a molecular weight of about 6,000 having a tradename KF 6028 available from ShinEtsu Chemical. Highly preferred alkyl dimethicone copolyols include cetyl dimethicone copolyol and stearyl dimethicone copolyol. A highly preferred commercially available alkyl dimethicone copolyol includes cetyl dimethicone copolyol, also called Methylpolysiloxane Cetylmethylpolysiloxane Poly(oxyethylene oxypropylene) Methylpolysiloxane Copolymer, having an HLB of about 5 and a molecular weight of about 13,000 having a tradename ABIL EM90 available from Goldschmidt Personal Care.

In a preferred embodiment, the water-in-oil emulsifier is a mixture of at least one ester-type surfactant and at least one silicone-type surfactant to provide a stable emulsion for the other essential components of the present invention.

### Oil-in-Water Emulsifier

The oil-in-water emulsion product for composition of the present invention comprises an oil-in-water emulsifier in an amount of preferably from about 0.1% to about 10%. When incorporated in solid oil-in-water emulsion forms, the amount included is preferably from about 1% to about 5%. A wide variety of emulsifiers can be employed herein. Known or conventional emulsifiers can be used in the composition, provided that the selected emulsifying agent is chemically and physically compatible with essential components of the composition, and provides the desired dispersion characteristics.

Non-limiting examples of oil-in-water emulsifiers useful herein are various non-ionic and anionic emulsifiers such as sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, soaps, and mixtures thereof.

Nonlimiting examples of other emulsifiers for use herein include: polyethylene glycol 20 sorbitan monolaurate (polysorbate 20), polyethylene glycol 5 soya sterol, steareth-20, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, polyoxyethylene 20 sorbitan trioleate (polysorbate 85), sorbitan monolaurate, polyoxyethylene 4 lauryl ether sodium stearate, polyglyceryl-4 isostearate, hexyl laurate, PPG-2 methyl glucose ether distearate, ceteth-10, diethanolamine cetyl phosphate, glyceryl stearate, PEG 40 hydrogenated castor oil, PEG-60 hydrogenated castor oil, and mixtures thereof.

Polyoxyalkylene hydrogenated castor oils useful herein include, for example, polyoxyethylene hydrogenated castor oils having 20-100 moles of ethylene oxides, such as polyoxyethylene (20) hydrogenated castor oil, polyethylene (40) hydrogenated castor oil, and polyoxyethylene (100) hydrogenated castor oil. Polyglycerin alkyl esters having the C10-20 of alkylsubstitute useful herein include, for example, those having 6-10 moles of glycerin units, such as polyglyceryl-6 laurate, polyglyceryl-10 laurate, and polyglyceryl-10 stearate. Polysorbates useful herein include, for example, those having 20-80 moles of ethylene oxides, such as polysorbate-20, polyborbate-40, polysorbate-60, and polysorbate-80. Polyethylene sterols and polyethylene hydrogenated sterols useful herein include, for example, those having 10-30moles of ethylene oxides, such as polyethylene (10) phytosterol, polyethylene (30) phytosterol, and polyethylene (20) cholesterol. Among the above nonionic surfactants, preferred are polysorbates, and more preferred are polysorbate-20, polysorbate-40, and mixtures thereof.

### Solid Wax

The composition of the present invention may comprise a solid wax for providing the aforementioned water-in-oil and oil-in-water emulsions in solid form. The solid water-in-oil emulsion and oil-in-water compositions of the present invention preferably comprise, by weight of the entire composition, from about 1% to about 5% of solid wax. Without being bound by theory, the species and levels of the solid wax herein is believed to provide consistency to the composition and coverage to the skin, while not negatively contributing to the spreadability upon application to the skin, and fresh and light feel of the skin.

The solid waxes useful herein are paraffin wax, microcrystalline wax, ozokerite wax, ceresin wax, carnauba wax, candellila wax, eicosanyl behenate, and mixtures thereof. A mixture of waxes is preferably used.

Commercially available solid waxes useful herein include: Candelilla wax NC-1630 available from Cerarica Noda, Ozokerite wax SP-1021 available from Strahl & Pitsh, and Eicosanyl behenate available from Cas Chemical.

### Additional Components

The composition of the present composition may further comprise a skin benefit agent dissolved or dispersed in the water phase, the oil component, or the powder components. When included, the skin benefit agent is included in an amount that does not affect the stability of the composition, typically by weight of the composition, at from about 0.001% to about 20%. The skin benefit agents useful herein include skin lightening agents, anti-acne agents, emollients, non-steroidal anti-inflammatory agents, topical anaesthetics, artificial tanning agents, antiseptics, anti-microbial and anti-fungal actives, skin soothing agents, UV protection agents, skin barrier repair agents, anti-wrinkle agents, anti-skin atrophy actives, lipids, sebum inhibitors, sebum inhibitors, skin sensates, protease inhibitors, skin tightening agents, anti-itch agents, hair growth inhibitors, desquamation enzyme enhancers, anti-glycation agents, antiperspirant actives, oxidative hair colorants, hair styling agents, and mixtures thereof.

The compositions hereof may further contain additional components such as are conventionally used in topical products, e.g., for providing aesthetic or functional benefit to the composition or personal surface, such as sensory benefits relating to appearance, smell, or feel, therapeutic benefits, or prophylactic benefits (it is to be understood that the above-described required materials may themselves provide such benefits). When included, the amount is kept to no more than about 10% by weight of the composition.

Examples of suitable topical ingredient classes include: powders and pigments that do not meet the definition of other powders described above including spherical powders that are not the water repelling silicone elastomer powder, anti-chelating agents, abrasives, astringents, dyes, essential oils, fragrance, film forming polymers, solubilizing agents, anti-caking agents, antifoaming agents, binders, buffering agents, bulking agents, denaturants, pH adjusters, propellants, reducing agents, sequestrants, cosmetic biocides, and preservatives.

### Examples

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

The following are foundation compositions of various product forms, method of preparation thereof, and technical and sensory assessment of their characteristics thereof. Examples 1-13 are those according to the present invention, while Comparative Examples 1-2 are those that are not according to the present invention. Further, Reference Examples 1 and 2 are provided for characterizing the preferred water repelling silicone elastomer powder herein.

### Reference Example 1

The water repelling silicone elastomer powder utilized in Examples below are prepared as such.

In a 1 liter glass beaker, 500 g of methylvinylpolysiloxane of formula (1) having a viscosity of 580 mm²/s and 19 g of methylhydrogenpolysiloxane of formula (2) having a viscosity of 30 mm²/s (namely, an amount wherein the number of hydrosilyl group is 1.06 per every olefin unsaturated group) were dissolved by mixing via a homomixer at 2000 rpm. Then, 3 g of polyoxyethylenelaurylether (9 mols of added ethyleneoxide) and 55 g of water was added and mixed with a homomixer at 6000 rpm to achieve an oil-in-water emulsion form and viscosifying, and further mixed for 15 minutes. Then, by adding 421 g of water under mixing at 2000 rpm, a homogenous white emulsion was obtained. This emulsion was transferred to a 1 liter glass flask having a mixing apparatus with an anchor mixing blade, adjusted to a temperature of 15-20° C., added with a co-solution of 0.8 g of toluene solution of chloroplatinic acid olefin complex (having platinum content of 0.5%) and 1.6 g of polyoxyethylenelaurylether (9 mols of added ethyleneoxide), and mixed at the same temperature for 12hrs, to obtain a water dispersion of fine particles of silicone elastomer. The silicone elastomer fine particles were spherical in shape by observing by optical microscope, and had a volume average particle size of 5 μm by measuring with an electric resistance method particle distribution measuring device “Multisizer-3” (Beckman Coulter).

870 g of such obtained water dispersant of spherical silicone elastomer fine particles were transferred to a 3 liter glass flask having a mixing apparatus with an anchor mixing blade, and added with 2013 g of water and 57 g of 28% ammonia solution. The pH of this fluid was 11.3. After adjusting the temperature to 5-10° C., 60 g of methyltrimethoxysilane (for 100 weight parts of spherical elastomer fine particle, 6.5 weight parts of hydrolytically condensed polymethylsilsesquioxane) was dropped over a period of 20minutes while keeping the fluid temperature at 5-10° C., mixed at the same temperature for another 1 hr, to complete the hydrolytic condensation of methyltrimethoxysilane.

The hydrolytic condensate fluid of methyltrimethoxysilane in the water dispersion of silicone elastomer fine particle was dehydrated with a pressurized filter to water content of about 30%. The dehydrate was transferred to a 5liter glass flask having a mixing apparatus with an anchor mixing blade, added with 3000 g of 50% methanol solution and mixed for 30 minutes, and dehydrated with a pressurized filter. The dehydrate was transferred to a 5 liter glass flask having a mixing apparatus with an anchor mixing blade, added with 3000 g of water and mixed for 30 minutes, and dehydrated with a pressurized filter. The dehydrate was dried at 105° C. in a hot air convention drier and crushed in a jet mill, to obtain a fluid fine particle. By observing with an electronic microscope, it was confirmed that the obtained was a spherical fine particle surface coated with particulates of about 100 nm, wherein the spherical silicone elastomer fine particle was coated with polymethylsilsequioxane. By dispersing the fine particles in water using surfactant and measured by measuring with an electric resistance method particle distribution measuring device “Multisizer-3” (Beckman Coulter), the volume average particle size was 5 μm. When measured by JIS K 6253, the obtained fine particles had a Durometer A Hardness of 29. When 1 g of the obtained fine particles were placed in an 100 ml beaker with 50 g of water and mixed for 1 minutes with a glass rod, none of the particles dispersed in water, but remained floating at the surface.

### Reference Example 2

The water repelling silicone elastomer powder utilized in Examples below are prepared as such.

Water dispersant of spherical silicone elastomer fine particles were obtained in the same manner as Reference Example 1.

870 g of such obtained water dispersant of spherical silicone elastomer fine particles were transferred to a 3 liter glass flask having a mixing apparatus with an anchor mixing blade, and added with 2013 g of water and 57 g of 28% ammonia solution. The pH of this fluid was 11.3. After adjusting the temperature to 5-10° C., 46.8 g of methyltrimethoxysilane (for 100 weight parts of spherical elastomer fine particle, 5.1 weight parts of hydrolytically condensed polymethylsilsesquioxane) was dropped over a period of 20 minutes while keeping the fluid temperature at 5-10° C., then 8.4 g of trimethylsilanol (for 100 weight parts of spherical elastomer fine particle, 1.9 weight parts of hydrolytically condensed polymethylsilsesquioxane) and 4.8 g of tetramethoxysilane (0.34 mols per 1 mol of trimethylsilanol) was dropped over a period of 5minutes while keeping the fluid temperature at 5-10° C., mixed at the same temperature for another 1 hr, to complete the hydrolytic condensation of methyltrimethoxysilane, tetramethoxysilane, and trimethylsilanol.

The hydrolytic condensate fluid of methyltrimethoxysilane, tetramethoxysilane, and trimethylsilanol methoxysilyl in the water dispersion of silicone elastomer fine particle was dehydrated with a pressurized filter to water content of about 30%. The dehydrate was transferred to a 5 liter glass flask having a mixing apparatus with an anchor mixing blade, added with 3000 g of 50% methanol solution and mixed for 30 minutes, and dehydrated with a pressurized filter. The dehydrate was transferred to a 5 liter glass flask having a mixing apparatus with an anchor mixing blade, added with 3000 g of water and mixed for 30 minutes, and dehydrated with a pressurized filter. The dehydrate was dried at 105° C. in a hot air convention drier and crushed in a jet mill, to obtain a fluid fine particle. By observing with an electronic microscope, it was confirmed that the obtained was a spherical fine particle surface coated with particulates of about 100 nm, wherein the spherical silicone elastomer fine particle was coated with polymethylsilsequioxane. By dispersing the fine particles in water using surfactant and measured by measuring with an electric resistance method particle distribution measuring device “Multisizer-3” (Beckman Coulter), the volume average particle size was 5 μm. When measured by JIS K 6253, the obtained fine particles had a Durometer A Hardness of 29.

When 1 g of the obtained fine particles were placed in an 100 ml beaker with 50 g of water and mixed for 1 minutes with a glass rod, none of the particles dispersed in water, but remained floating at the surface.

TABLE 1
Compositions for Examples 1-3 water-containing
capsule product forms and test results

| | Components | Ex. 1 | Ex. 2 | Ex. 3 |
|---|---|---|---|---|
| A | Vinyl Dimethicone/Methicone Silsesquioxane Crosspolymer | | 10 | |
| | of Reference Example 1 | | | |
| A | Trimethylsilyl Vinyl Dimethicone/Methicone Silsesquioxane | 10 | | 20 |
| | Crosspolymer of Reference Example 2 | | | |
| A | Titanium Dioxide coated with Triethoxycaprylylsilane | 1 | 1 | |
| | (250 nm) *1 | | | |
| A | Titanium Dioxide coated with Triethoxycaprylylsilane | 13 | 13 | |
| | (10 nm/60 nm) *2 | | | |
| A | Silica Dimethyl Silylate (15 nm) *3 | | | 2.5 |
| A | Mica coated with Triethoxycaprylylsilan (20 μm) *4 | 1.87 | 1.87 | 2.87 |
| A | Yellow Iron Oxide coated with triethoxycaprylylsilane | 0.35 | 0.35 | 0.35 |
| | (400 nm) *5 | | | |
| A | Black Iron Oxide coated with triethoxycaprylylsilane (400 nm) | 0.1 | 0.1 | 0.1 |
| | *6 | | | |
| A | Red Iron Oxide coated with Triethoxycaprylylsilane (400 nm) | 0.1 | 0.1 | 0.1 |
| | *7 | | | |
| A | DL-alpha-Tocopheryl Acetate containing Silica coated with | 0.2 | 0.2 | 0.2 |
| | Dimethicone (5 μm) *8 | | | |
| A | Fragrance | | | 0.01 |
| B | Sodium Carboxymethyl Starch *9 | 0.5 | 0.5 | 0.5 |
| B | Glycerin | 15 | 15 | 15 |
| B | Niacinamide *10 | 3.5 | 3.5 | 3.5 |
| B | DL-Panthenol *11 | 1.12 | 1.12 | 1.12 |
| B | Preservative | 0.7 | 0.7 | 0.7 |
| B | DE-IONIZED WATER | 52.56 | 52.56 | 53.05 |
| | Total | 100 | 100 | 100 |
| | Capsulation | Good | Good | Good |
| | Shock Stability | 12 | 9 | 8 |
| | Cooling Sensory on Application | 4.4 | 4.2 | 3.2 |

TABLE 2
Compositions for Comparative Examples 1-2 and test results

| | Name | Com. Ex. 1 | Com. Ex. 2 |
|---|---|---|---|
| A | Trimethylsilyl Vinyl Dimethicone/Methicone Silsesquioxane | | 10 |
| | Crosspolymer of Reference Example 2 | | |
| A | Vinyl Dimethicone/Methicone Silsesquioxane Crosspolymer *12 | 10 | |
| A | Titanium Dioxide coated with Triethoxycaprylylsilane | 13 | |
| | (10 nm/60 nm) *2 | | |
| A | Titanium Dioxide coated with Triethoxycaprylylsilane | 1 | 1 |
| | (250 nm) *1 | | |
| A | Yellow Iron Oxide coated with Triethoxycaprylylsilane | 0.35 | 0.35 |
| | (400 nm) *5 | | |
| A | Black Iron Oxide coated with Triethoxycaprylylsilane | 0.1 | 0.1 |
| | (400 nm) *6 | | |
| A | Red Iron Oxide coated with Triethoxycaprylylsilane (400 nm) *7 | 0.1 | 0.1 |
| A | DL-alpha-Tocopheryl Acetate containing Silica coated with | 0.2 | 0.2 |
| | Dimethicone (5 μm) *8 | | |
| A | Mica coated with Triethoxycaprylylsilan (20 μm) *4 | 1.87 | 14.87 |
| B | Sodium Carboxymethyl Starch *9 | 0.5 | 0.5 |
| B | Glycerin | 15 | 15 |
| B | Niacinamide *10 | 3.5 | 3.5 |
| B | DL-Panthenol *11 | 1.12 | 1.12 |
| B | Preservative | 0.7 | 0.7 |
| B | DE-IONIZED WATER | 52.56 | 52.56 |
| | Total | 100 | 100 |
| | Capsulation | Good | Not Good |
| | Shock Stability | 4 | N/A |
| | Cooling Sensory on Application | 4.6 | N/A |

Definitions of Components for Examples 1-3 and Comparative Examples 1-2
*1 Titanium Dioxide coated with Triethoxycaprylylsilane (250 nm): OTS-2 TiO2 CR-50 available from Daito Kasei.
*2 Titanium Dioxide coated with Triethoxycaprylylsilane (10/60 nm): OTS-11 TTO-V-3 available from Daito Kasei.
*3 Silica Dimethyl Silylate (15 nm): Aerosil R 972 available from Nihon Aerosil.
*4 Mica coated with Triethoxycaprylylsilan (20 μm): OTS-2 MICA Y-2300 available from Daito Kasei.
*5 Yellow Iron Oxide coated with Triethoxycaprylylsilane (400 nm): OTS-2 YELLOW LL-100P available from Daito Kasei.
*6 Black Iron Oxide coated with Triethoxycaprylylsilane (400 nm): OTS-2 BLACK BL-100P available from Daito Kasei.
*7 Red Iron Oxide coated with Triethoxycaprylylsilane (400 nm): OTS-2 RED R-516P available from Daito Kasei.
*8 DL-alpha-Tocopheryl Acetate containing Silica coated with Dimethicone (5 μm): SA-SB-705/VEAC(50%) available from Miyoshi Kasei.
*9 Sodium Carboxymethyl Starch: COVAGEL available from LCW.
*10 Niacinamide: Niacinamide USP available from DSM.
*11 DL-Panthenol: D-Panthenol USP, available from DSM
*12 Vinyl Dimethicone/Methicone Silsesquioxane Crosspolymer (5 μm, Durometer A Hardness: 30): KSP-100 available from ShinEtsu.

### Method of Preparation for Examples 1-3 and Comparative Example 1-2

Components A are mixed and transferred to a container that has a hydrophobic inner surface. Components B are separately mixed and transferred to the same container. The container is closed and shook by TURBLER Shaker Mixer T2F (Willy A. Bachofen AG) at 95 rpm for 3 min.

### Methods of Tests for Examples 1-3 and Comparative Examples 1-2

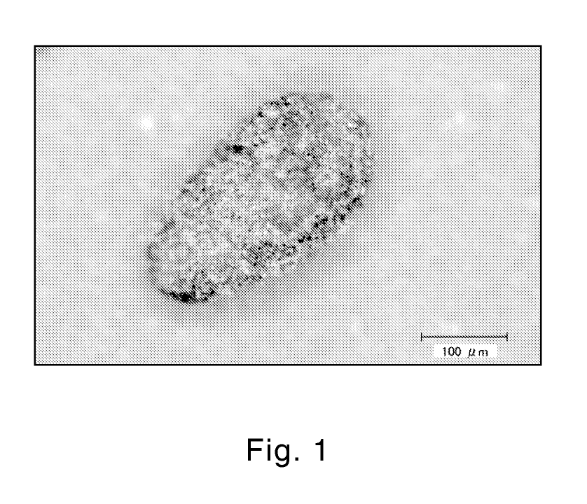

Capsulation: If capsules of even fine particles are observed by DIGITAL MICROSCOPE VHX-900 from KEYENCE, evaluation is “Good”. If the capsules are not formed, evaluation is “Not Good”. FIG. 1 provides a microscopic photograph at a magnitude of 200 times of a capsule of a preferred embodiment of the present invention that has been successfully formed. As can be seen from FIG. 1, a clear boundary of the capsule is observed. When the capsule is not formed, such boundary is not observed, but rather a more or less homogenous mass is observed. For those compositions that did not form capsules, it is not possible to conduct the remaining tests.

Shock Stability (Tumbling Impact Method): 5 g of powder sample is weighed and placed in a 50 ml Poly Propylene container. After closing a cap, put the container into 1 L plastic container. The 1 L container is capped and set on a TURBLER Mixer Type T2F (Willy A. Bachofen AG), and shook at 100 rpm for 1 min, and stopped for observation. The same shaking and observation procedure is repeated after each minute of shaking until a total of 15 cycles. If the powder sample is collapsed and changed to liquid, it is considered end point and total shaking time is recorded. If the sample endures shaking for total 5 minutes and collapsed at total 6 minutes, the value is defined as “5 minutes”. Those compositions enduring 8 minutes of shaking are considered as having acceptable stability.

Cooling Sensory on Application: Cooling Sensory is evaluated upon application on the hand by five expert panelists with 5 scale grades (No Cooling—1, Very Weak Cooling—2, Weak Cooling—3, Strong Cooling—4 and Very Strong Cooling—5). Then average is calculated. Those compositions that do not provide more than a calculated score of 3.0 are considered as not providing satisfactory cooling sensation.

### Evaluation of Examples 1-3 and Comparative Examples 1-2

The results of Examples 1-3 and Comparative Examples 1-2 are found in Tables 1 and 2. Comparative Example 1 which is devoid of the water repelling silicone elastomer powder, and containing a conventional silicone elastomer powder of similar hardness, did not provide acceptable stability. Comparative Example 2 having less than required amount of the filler powder did not form a capsule.

### Usage of Examples 1-3

The capsules of Examples 1-3 are useful as collapsible water-containing capsules having appropriate shock stability such that it is stable under normal storage conditions as well as normal mixing processes, however, collapses upon a certain shear stress upon application on the skin. When collapsed, the capsules of Examples 1-3 provide good feel and good appearance on the skin by balanced coverage and natural look, as well as good wear.

TABLE 3
Compositions for Examples 4-5 loose powder product forms

| | | Ex. 4 | Ex. 5 |
|---|---|---|---|
| A | Mica | 38.45 | 38.45 |
| A | Vinyl Dimethicone/Methicone Silesquioxane | 50 | |
| | Crosspolymer of Reference Example 1 | | |
| A | Trimethylsilyl Vinyl Dimethicone/Methicone | | 50 |
| | Silsesquioxane Crosspolymer of | | |
| | Reference Example 2 | | |
| A | Titanium Dioxide | 5 | 5 |
| A | Methylparaben | 0.2 | 0.2 |
| A | Propylparaben | 0.1 | 0.1 |
| A | Imidazolidinyl Urea | 0.25 | 0.25 |
| B | Red iron oxide | 1 | 1 |
| B | Yellow iron oxide | 5 | 5 |
| | Total | 100 | 100 |

### Method of Preparation for Examples 4-5

Components A are milled together until fully dispersed. Components B are added to A and blended until uniform.

TABLE 4
Compositions for Examples 6-7 pressed powder product forms

| | | Ex. 6 | Ex. 7 |
|---|---|---|---|
| A | Soft Talc | 32.7 | 32.7 |
| A | Pyrenean Silk Talc | 45.214 | 45.214 |
| A | Titanium Dioxide | 2 | 2 |
| A | Silk Mica | 4 | 4 |
| A | Vinyl Dimethicone/Methicone Silesquioxane | 2 | |
| | Crosspolymer of Reference Example 1 | | |
| A | Trimethylsilyl Vinyl Dimethicone/Methicone | | 2 |
| | Silsesquioxane Crosspolymer of | | |
| | Reference Example 2 | | |
| A | Methylparaben | 0.3 | 0.3 |
| A | Propylparaben | 0.1 | 0.1 |
| A | Sodium Dehyrdroacetate Monohydrate | 0.1 | 0.1 |
| A | Iron Oxide (Yellow) | 0.622 | 0.622 |
| A | Iron Oxide (Black) | 0.182 | 0.182 |
| A | Iron Oxide (Red) | 0.272 | 0.272 |
| A | Red 36 | 0.2 | 0.2 |
| A | Yellow 5 Aluminum Lake | 0.3 | 0.3 |
| B | Octyldodecyl Stearoyl Stearate | 2.67 | 2.67 |
| B | Hydrogenated Coco-glycerides | 2.67 | 2.67 |
| B | Silicone Oil 350 centistoke | 6.67 | 6.67 |
| | Total | 100 | 100 |

### Method of Preparation for Examples 6-7

Phase A ingredients are bulk mixed in a ribbon blender or double cone blender. Once the bulk Phase A ingredients are homogenous, they are passed through a hammer mill to break up powder agglomerates and extend the inorganic pigments. In parallel, the Phase B binders are heated to 60° C. On completion of milling, Phase A is returned to the ribbon blender and the hot Phase B binders are added and mixed into the bulk powder. Once the Phase A and B mixture is homogenous, the combined powder and binder ingredients are passed through a Comil. The powder is then pressed into its final form.

TABLE 5
Compositions for Examples 8-9 water-in-oil product forms

| | | Ex. 8 | Ex. 9 |
|---|---|---|---|
| A1 | Cyclopentacyloxane and dimethicone copolyol | 9 | 9 |
| A2 | Tridecyl Neopentanoate | 6.3 | 6.3 |
| A3 | Decamethylcyclopentacyloxane | 14.543 | 14.543 |
| A4 | Polyethylene Glycol (7) Lauryl Ether | 0.5 | 0.5 |
| A5 | Propylparaben | 0.15 | 0.15 |
| B1 | Titanium Dioxide (And) Polyglyceryl-4 Isostearate (And) Cetyl | 12.062 | 12.062 |
| | Dimethicone Copolyol (And) Hexyl Laurate (And) Isopropyl | | |
| | Titanium Triisostearate *1 | | |
| B2 | Iron Oxide (CI 77492) (And) Polyglyceryl-4 Isostearate (And) Cetyl | 1.382 | 1.382 |
| | Dimethicone Copolyol (And) Hexyl Laurate (And) Isopropyl | | |
| | Titanium Triisostearate *1 | | |
| B3 | Iron Oxide (CI 77491) (And) Polyglyceryl-4 Isostearate (And) Cetyl | 0.314 | 0.314 |
| | Dimethicone Copolyol (And) Hexyl Laurate (And) Isopropyl | | |
| | Titanium Triisostearate *1 | | |
| B4 | Iron Oxide (CI 77499) (And) Polyglyceryl-4 Isostearate (And) Cetyl | 0.189 | 0.189 |
| | Dimethicone Copolyol (And) Hexyl Laurate (And) Isopropyl | | |
| | Titanium Triisostearate *1 | | |
| B5 | Vinyl Dimethicone/Methicone Silsesquioxane Crosspolymer of | 5 | |
| | Reference Example 1 | | |
| B6 | Trimethylsilyl Vinyl Dimethicone/Methicone Silsesquioxane | | 5 |
| | Crosspolymer of Reference Example 2 | | |
| C1 | Deionized water | 17.51 | 17.51 |
| C2 | Polyvinylpyrrolidone | 1.5 | 1.5 |
| C3 | Phenoxyethanol | 0.25 | 0.25 |
| C4 | Trisodium edetate Edetate | 0.1 | 0.1 |
| C5 | Sodium Chloride | 1 | 1 |
| C6 | Sodium dehydroacetate monohydrate | 0.2 | 0.2 |
| D1 | Cyclopentasiloxane (and) C30-45 Alkyl Cetearyl Dimethicone | | 12 |
| | Crosspolymer *2 | | |
| D2 | Cyclopentasiloxane (and) C30-45 Alkyl Cetearyl Dimethicone | 30 | 18 |
| | Crosspolymer (and) titanium dioxide (and) iron oxides *3 | | |
| | Total | 100 | 100 |

*1 Tradename ITT Coated Pigments available from Kobo Products
*2 Tradename Velvesil 125 available from General Electric Silicone
*3 Tradename 1111-21-937 available from General Electric Silicone

### Method of Preparation for Examples 8-9

Combine Phase C in plastic bucket. Provide maximum prop mixer blending without air incorporation. Add Phase A ingredients 1-5 to stainless steel jacketed vessel and begin high shear mixing. Add Phase B ingredients to Phase A and begin milling on HIGH for approximately 30 minutes. Add phase C to phase AB in vessel with homogenization. Continue homogenizing until batch uniformity is visually achieved. Add Phase D ingredients and homogenize until uniformity is achieved

TABLE 6
Compositions for Examples 10-11 oil-in-water product forms

| | | Ex. 10 | Ex. 11 |
|---|---|---|---|
| A1 | Decamethylcyclopentasiloxane | 9.145 | 9.145 |
| A2 | Dodecamethyl cyclohexasiloxane | 2.065 | 2.065 |
| A3 | Tridecyl Neopentanoate | 8 | 8 |
| A4 | PCA Dimethicone | 2 | 2 |
| A5 | Propylparaben | 0.15 | 0.15 |
| A6 | Arachadyl Behenate | 0.3 | 0.3 |
| A7 | Stearyl Alcohol | 0.75 | 0.75 |
| B1 | Titanium Dioxide (And) Polyglyceryl-4 Isostearate (And) Cetyl | 9.075 | 9.075 |
| | Dimethicone Copolyol (And) Hexyl Laurate (And) Isopropyl | | |
| | Titanium Triisostearate *1 | | |
| B2 | Iron Oxide (CI 77492) (And) Polyglyceryl-4 Isostearate (And) | 0.81 | 0.81 |
| | Cetyl Dimethicone Copolyol (And) Hexyl Laurate (And) Isopropyl | | |
| | Titanium Triisostearate *1 | | |
| B3 | Iron Oxide (CI 77491) (And) Polyglyceryl-4 Isostearate (And) | 0.262 | 0.262 |
| | Cetyl Dimethicone Copolyol (And) Hexyl Laurate (And) Isopropyl | | |
| | Titanium Triisostearate *1 | | |
| B4 | Iron Oxide (CI 77499) (And) Polyglyceryl-4 Isostearate (And) | 0.143 | 0.143 |
| | Cetyl Dimethicone Copolyol (And) Hexyl Laurate (And) Isopropyl | | |
| | Titanium Triisostearate *1 | | |
| B5 | Vinyl Dimethicone/Methicone Silesquioxane of Reference | 2 | |
| | Example 1 | | |
| B6 | Trimethylsilyl Vinyl Dimethicone/Methicone Silsesquioxane | | 2 |
| | Crosspolymer of Reference Example 2 | | |
| C1 | Deionized Water | 52 | 52 |
| C2 | Methylparaben | 0.2 | 0.2 |
| C3 | Phenoxyethanol | 0.5 | 0.5 |
| C4 | Hydroxypropyl Starch Phosphate | 2 | 2 |
| C5 | Glycerin | 2.25 | 2.25 |
| C6 | Butylene Glycol | 2.25 | 2.25 |
| C7 | Polyvinylpyrrolidone | 1 | 1 |
| C8 | Trisodium Edetate | 0.1 | 0.1 |
| C9 | Sucrose Palmitate (and) Glyceryl Stearate (and) Glyceryl Stearate | 2 | 2 |
| | Citrate (and) Sucrose (and) Mannan (and) Xanthan Gum *2 | | |
| C10 | Red Pigment | 1 | 1 |
| C11 | Yellow Pigment | 1.5 | 1.5 |
| C12 | Blue Pigment | 0.5 | 0.5 |
| | Total | 100 | 100 |

*1 Tradename ITT Coated Pigments available from Kobo Products
*2 Tradename Arlatone V-175 available from Uniquima

### Method of Preparation for Examples 10-11

Combine ingredients C1 and C9 with maximum propeller. Add Phase C ingredients 2, 3, 5, 6, 7, 8, 10, 11, and 12. Provide maximum prop mixer blending without air incorporation. Heat Phase C to 70-80° C. Once batch reaches 70-80° C. add 50% C4. Add Phase A ingredients 1-5 to separate vessel and begin homogenizing batch. Heat Phase A to 70-80° C. Add Phase B to Phase A shear on HIGH for approximately 20-30 minutes. Once Phase AB reaches 70-80° C. add Phase A ingredients 6-7. Transfer Phase AB to Phase C while prop mixing. Blend until uniform in appearance. Homogenize batch with high shear. Add remaining 50% C4. Maintain until uniformity is achieved.

TABLE 7
Compositions for Example 12-13 solid water-in-oil product forms

| | | Ex. 12 | Ex. 13 |
|---|---|---|---|
| A | Isotridecyl Isononanoate *1 | 6 | 6 |
| A | Decamethylcyclopentasiloxane *2 | 2.8 | 25.6 |
| A | Lauryl PEG-9 Polydimethyl-siloxyethyl Dimethicone *3 | 1.5 | 1.5 |
| A | Slurry of Iron Oxide, Cyclopentasiloxane, Dimethicone and | 0.5 | 2 |
| | Disodium Hydrogenated Glutamate *4 | | |
| A | Mixture of ascorbyl tetraisopalmitate, silica, and dimethicone *5 | 1 | 0.1 |
| A | Powder Mix *6 | 0.5 | 0.1 |
| A | Mixture of mica, titanium dioxide, silica, iron oxide, alumina, and | 0.5 | 0.1 |
| | dimethicone/methicone copolymer *7 | | |
| A | 2-ethylhexyl 4-methoxycinnamate *8 | 3 | 3 |
| A | 2-Hydroxy-4-methoxybenzophenone (Benzophenone-3) *9 | 0.5 | 0.5 |
| A | Titanium Dioxide and Methicone *10 | | 5.1 |
| A | Titanium Dioxide, Dimethicone, Aluminium Hydroxide and | 2 | |
| | Stearic Acid *11 | | |
| A | Cyclopentasiloxane (87.4%) and Dimethicone Crosspolymer | 26 | 5 |
| | (12.6%) Blend *12 | | |
| A | Isododecane (75%) and PEG-15/Lauryl Dimethicone Crosspolymer | 8 | 1 |
| | (25%) Blend *13 | | |
| A | Vinyl Dimethicone/Methicone Silesquioxane Crosspolymer of | 5 | |
| | Reference Example 1 | | |
| A | Trimethylsilyl Vinyl Dimethicone/Methicone Silsesquioxane | | 7 |
| | Crosspolymer of Reference Example 2 | | |
| B | Water | 21.5 | 25 |
| B | Preservatives | 0.7 | 0.7 |
| B | Phenylbenzimidazole Sulfonic Acid *14 | 1 | 3 |
| B | 2-Hydroxy-4-methoxybenzophenone-5-Sulfonic Acid | 0.5 | 0.5 |
| | (Benzophenone-4) *15 | | |
| B | Butylene Glycol *16 | | 7.4 |
| B | Glycerin *17 | 5 | |
| B | Niacinamide *18 | 1 | |
| B | Mixture of Saccharomycopsis Ferment Filtrate and Butylene | 5 | |
| | Glycol and Methylparaben *19 | | |
| B | Triethanolamine *20 | 2.5 | 2.5 |
| C | Candelilla Wax *22 | 3 | 2 |
| C | Ceresin *23 | 2.5 | 1.9 |
| | Total | 100 | 100 |

*1 Isotridecyl Isononanoate: Crodamol TN available from Croda
*2 Decamethylcyclopentasiloxane: SH245 available from Dow Corning
*3 Lauryl PEG-9 Polydimethyl-siloxyethyl Dimethicone: KF6038 available from Shinetsu Chemical Co., Ltd.
*4 Slurry of Iron Oxide, Cyclopentasiloxane, Dimethicone and Disodium Hydrogenated Glutamate: SA/NAI-Y-10/D5 (70%), SA/NAI-R-10/D5 (65%) and SA/NAI-B-10/D5 (75%) available from Miyoshi Kasei
*5 Mixture of ascorbyl tetraisopalmitate, silica, and dimethicone: SA-SB-705/VC-IP available from Miyoshi Kasei
*6 Powder Mix: Mixture of Methyl Methacrylate Crosspolymerand Sodium Cocoyl Glycinate and Calcium Hydroxide and Iron Oxides with tradename Grandeur Pearl Powder Pink available from Miyoshi Kasei
*7 Mixture of mica, titanium dioxide, silica, iron oxide, alumina, and dimethicone/methicone copolymer: Relief Color Pink P-2 available from Nihon Shokubai
*8 2-ethylhexyl 4-methoxycinnamate: PARSOL MCX available from Symrise
*9 2-Hydroxy-4-methoxybenzophenone (Benzophenone-3): available from BASF
*10 Titanium Dioxide and Methicone: SI-T-CR-50-Z (80%) LHC available from Miyoshi Kasei
*11 Titanium Dioxide, Dimethicone, Aluminum Hydroxide and Stearic Acid: SAST-UFTR-Z available from Miyoshi Kasei
*12 Cyclopentasiloxane (87.4%) and Dimethicone Crosspolymer (12.6%) Blend: DC-9040 available from Dow Corning
*13 Isododecane (75%) and PEG-15/Lauryl Dimethicone Crosspolymer (25%) Blend: KSG-320 available from Shinetsu Silicone
*14 Phenylbenzimidazole Sulfonic Acid: Neo Haliopan Hydro available from Symrise
*15 2-Hydroxy-4-methoxybenzophenone-5-Sulfonic Acid (Benzophenone-4): available from BASF
*16 Butylene Glycol: 1,3 Butylene Glycol available from Kyowa Hakko Kogyo
*17 Glycerin: Glycerin USP available from Asahi Denka
*18 Niacinamide: Niacinamide available from Reilly Industries Inc.
*19 Mixture of Saccharomycopsis Ferment Filtrate and Butylene Glycol and Methylparaben: SK-2 4X available from P&G
*20 Triethanolamine: TEA available from Dow Chemical
*21 Candelilla Wax: Candelilla wax NC-1630 available from Cerarica Noda
*22 Ceresin: Ozokerite wax SP-1021 available from Strahl & Pitsh

### Method of Preparation for Examples 12-13

1) Components of Phase A are mixed with suitable mixer until homogeneous to make a lipophilic mixture. 2) Components of Phase B are dissolved with suitable mixer until all components are completely dissolved to make a water phase. Phase B is added into the product of step 1) to make emulsion at room temperature using homogenizer. 3) Components of phase C are heated to dissolve at 80-85° C. in a sealed tank. Phase C is added into the product of step 2) using homogenizer. 4) Finally, the obtained emulsion is filled in an air-tight container and allowed to cool to room temperature using a cooling unit.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A cosmetic foundation composition comprising:
(a) from about 0.1% to about 85% of a water repelling silicone elastomer powder comprising 100 weight parts of a spherical silicone elastomer particle and 0.5-25 weight parts of polyorganosilsequioxane for coating the spherical silicone elastomer particle; wherein the water repelling silicone elastomer powder does not disperse in, but floats in water; has an average particle size of at least 1 μm and has a softness of from about 10 to about 80 measured by Durometer A Hardness; and
(b) a suitable carrier, the suitable carrier comprising from about 0.1% to about 99.8% by weight of the composition of a powder component.

2. The foundation of claim 1 wherein the surface of the coated polyorganosilsequioxane of the water repelling silicone elastomer powder is further bonded with a trimethylsilyl group.

3. The foundation of claim 1 wherein the surface coated polyorganosilsequioxane of the water repelling silicone elastomer powder is further condensated by hydrolyzing with tetraalkoxysilane and at least one silylation agent selected from the group consisting of trimethylalkoxysilane, trimethylsilanol, and hexamethyldisilazine.

4. The composition of claim 1 in the form of a collapsible water-containing capsule wherein the carrier comprises, by weight of the composition:
(a) from about 0.1 to about 60% of a powder component wherein the total of the water repelling silicone elastomer powder and the powder component is at least 5%; and
(b) from about 40% to about 95% of a water phase.

5. The composition of claim 1 in the form of a loose powder wherein the carrier comprises, by weight of the composition, from about 15% to about 99.8% of a powder component.

6. The composition of claim 1 in the form of a pressed powder wherein the carrier comprises, by weight of the composition:
(a) from about 55% to about 98.9% of a powder component; and
(b) from about 1% to about 25% of a liquid binder selected from a water phase, a liquid oil, a water-in-oil emulsifier, and mixtures thereof.

7. The composition of claim 1 in the form of a water-in-oil emulsion wherein the carrier comprises, by weight of the composition:
(a) from about 5% to about 60% of an oil component;
(b) from about 0.1% to about 25% of a water-in-oil emulsifier; and
(c) from about 5% to about 70% of a water phase.

8. The composition of claim 1 in the form of an oil-in-water emulsion wherein the carrier comprises, by weight of the composition:
(a) from about 5% to about 60% of an oil component;
(b) from about 0.1% to about 25% of a oil-in-water emulsifier; and
(c) from about 10% to about 85% of a water phase.

9. The composition of claim 7 or 8 in the form of a solid emulsion wherein the carrier further comprises, by weight of the composition, from about 0.1% to about 10% of a solid wax
